(19) United States
(12) Patent Application Publication
(10) Pub. No.: US 2006/0147934 A1
(43) Pub. Date: Jul. 6, 2006

(54) High speed assay for glycosyl transferases

(76) Inventors: Matt S. Anderson, ChathamTownship, NJ (US); Sheryl A. Hyland, Rahway, NJ (US)

Correspondence Address: MERCK AND CO., INC, P O BOX 2000, RAHWAY, NJ, 07065-0907, US

(21) Appl. No.: 10/544,700

(22) PCT Filed: Feb. 2, 2004
(86) PCT No.: PCT/US04/02922

§ 371 Date: Aug. 5, 2005

Related U.S. Application Data
(60) Provisional application No. 60/445,301, filed on Feb. 5, 2003.

(51) Int. Cl. C12Q 1/68; C12Q 1/48

(52) U.S. Cl. 435/6; 435/15

(57) ABSTRACT

The polyprenyl phosphate: N-acetylhexosamine-1-phosphate transferase family are glycosyl transferase enzymes of interest in the development of antibacterial treatments. The bacterial proteins WecA and MraY are exemplary transferases, each of which catalyzes the transfer of a specific hexosamine 1-P from a soluble UDP-hexosamine substrate to a polyprenyl phosphate carrier at the membrane surface. The present invention provides a generalizable, high throughput, one-pot assay for this type of enzymatic activity by incorporating a solid-liquid, bead-based separation system to selectively adsorb the highly hydrophobic products of the reaction. By judicious choice of radiolabeled UDP-hexosamine precursor, the assay format can be used to quantitate the products of diverse members of this transferase family as well as enzymes that catalyze the further modification of these transferase products. Thus, the use of this flexible assay format allows biochemical and enzymologic analysis of many such membrane bound transferases.

### CROSS-REFERENCE TO RELATED APPLICATIONS

xxxx

### STATEMENT REGARDING FEDERALLY-SPONSORED R&D

xxxxx

### REFERENCE TO MICROFICHE APPENDIX

Xxxxx

### FIELD OF THE INVENTION

The present invention relates to the field of assays in which the products of glycosyl transferases are detected or measured.

### BACKGROUND OF THE INVENTION

The study of the murein biosynthetic pathway has its roots in the 1940s when Park and Johnson described the effects of penicillin on bacterial cell walls (Park, J. T. and Johnson, M. J. (1949) Accumulation of Labile Phosphate in Staphylococcus aureus Grown in the Presence of Penicillin. J. Biol. Chem. 179, 585-592.). Later Strominger and co-workers elucidated many other aspects of wall synthesis including the involvement of a lipid carrier in the pathway (Anderson J. S., Matsuhashi, M., Haskin, M. A. and Strominger J. L. (1965) Lipid-phosphoacetylmuramyl-pentapeptide and lipid-phosphodisaccharide-pentapeptide: presumed membrane transport intermediates in cell wall synthesis. Proc. Natl. Acad. Sci. U.S.A. 53, 881-889; Dietrich, C. P., Colucci, A. V. and Strominger J. L. (1967) Biosynthesis of the peptidoglycan of bacterial cell walls. V. Separation of protein and lipid components of the particulate enzyme from Micrococcus lysodeikticus and purification of the endogenous lipid acceptors. J. Biol. Chem. 242, 3218-3225.). This biosynthetic route has since been divided on functional grounds into three stages.

Stage I, cytoplasmic synthesis, builds precursors used as repetitive elements for wall synthesis. Stage II functionalizes these units on the membrane for final placement during Stage III where they reach growth sites and are placed into the wall (FIG. 1). The enzymes that collectively create the lipid-linked precursors are of particular interest as antibacterial targets given their membrane association and the relative paucity of bactoprenyl phosphate carrier available to a cell (van Heijenoort, Y., Gomez, M., Derrien, M., Ayala, J. and van Heijenoort, J. (1992) Membrane intermediates in the peptidoglycan metabolism of Escherichia coli: possible roles of PBP1b and PBP3. J. Bacteriol. 174, 3549-3557.) Indeed, since the discovery of penicillin, antibiotic substances have been discovered that inhibit each transformation catalyzed at the membrane surface (Gadebusch, H. H., Stapley, E. O., Zimmerman S. B. (1992) The discovery of cell wall active antibacterial antibiotics. Crit. Rev. Biotechnol. 12, 225-243.).

The first of these enzymes, the MraY translocase, is a member of a much larger family of integral membrane proteins that translocate the hexosamine 1-phosphate of a UDP-D-amino hexosamine donor to a membrane-associated polyprenyl phosphate. This broad family extends from bacterial murein, enterobacterial common antigen (Schmidt, G., Mayer, H., Makela, P. H. (1976) Presence of rfe genes in Escherichia coli: their participation in biosynthesis of O antigen and enterobacterial common antigen. J. Bacteriol. 127, 755-762.), O-antigen and capsule synthesis (Rick, P. D. and Silver, R. P. (1996) Enterobacterial common antigen and capsular polysaccharides, p. 104-122. In Neidhardt, F. C. and R. Curtiss (ed.), Escherichia coli and Salmonella: Cellular and Molecular Biology, 2nd. ed. ASM Press, Washington.), to the initial steps of N-linked glycoprotein biosynthesis in eukaryotes (Bugg, T. D. and Brandish, P. E. (1994) From peptidoglycan to glycoproteins: common features of lipid-linked oligosaccharide biosynthesis. FEMS Microbiol. Lett. 119, 255-262.).

This family can be further subdivided into several classes based on amino acid and sequence and substrate specificity, many details of which are only beginning to emerge (Lehrman, M. A. (1994) A Family of UDP-GlcNAc/MurNAc: polyisoprenol-P GlcNAc/MurNAc-1-P transferases. Glycobiology 4, 768-771; Anderson, M. S., Eveland, S. S., and Price, N. P. J. (2000) Conserved Cytoplasmic Motifs that Distinguish Sub-Groups of the Polyprenyl phosphate: N-Acetylhexosamine-1-Phosphate Transferase Family. FEMS Microbiol. Lett. 191, 169-175.).

Currently, many of these enzymes are assayed using extractions or paper chromatography, both of which can be tedious and low throughput. A problem exists in that extraction assays often suffer from high backgrounds making inhibition studies difficult. While progress has been made in the throughput of assays designed to measure the murein stage II enzymes as a target of antibiotic drug screening (Branstrom, A. A., Midha, S., Longley, C. B., Ham, K., Baizman, E. R. and Axelrod, H. R. (2000) Assay for Identification of Inhibitors for Bacterial MraY Translocase or MurG Transferase. Anal. Biochem. 280, 315-319; Chandrakala, B., Elias, B. C., Mehra, U., Umapathy, N. S., Dwarakanath, P., Balganesh, T. S. and DeSousa, S. M. (2001) Novel Scintillation Proximity Assay for Measuring Membrane-Associated Steps of Peptidoglycan Biosynthesis in Escherichia coli. Antimicrob. Agents Chemother. 45, 768-775.), there is a need for tools which can be generalized to assay many of these transferases with high reproducibility, low background signals and rapid throughput.

Several assay formats have been described for the assay of Stage II bacterial cell wall synthesis enzymes. Each measures radioactivity of a product bound to a bead, most commonly, by either the affinity of biotin-derivatized substrates for streptavidin (Branstrom, A. A., Midha, S., Longley, C. B., Ham, K., Baizman, E. R. and Axelrod, H. R. (2000) Assay for Identification of Inhibitors for Bacterial MraY Translocase or MurG Transferase. Anal. Biochen. 280, 315-319; Men, H., Park P., Ge., M. and Walker S. (1998) Substrate Synthesis and Activity Assay for MurG. J. Am. Chem. Soc. 120, 2484-2485.) or that of wheat germ agglutinin for GlcNAc (Chandrakala, B., Elias, B. C., Mehra, U., Umapathy, N. S., Dwarakanath, P., Balganesh, T. S. and DeSousa, S. M. (2001) Novel Scintillation Proximity Assay for Measuring Membrane-Associated Steps of Peptidoglycan Biosynthesis in Escherichia coli. Antimicrob. Agents Chemother. 45, 768-775.). While offering the advantages of throughput and homogeneity, they are restricted to either the murein enzyme reactions (Branstrom, A. A., Midha, S., Longley, C. B., Ham, K., Baizman, E. R. and Axelrod, H. R. (2000) Assay for Identification of Inhibitors for Bacterial MraY Translocase or MurG Transferase. Anal. Biochem. 280, 315-319; Men, H., Park P., Ge., M. and Walker S. (1998) Substrate Synthesis and Activity Assay for MurG. J. Am. Chem. Soc. 120, 2484-2485.) or to metabolic pathways where GlcNAc residues are sufficiently available for lectin capture (Chandrakala, B., Elias, B. C., Mehra, U., Umapathy, N. S., Dwarakanath, P., Balganesh, T. S. and DeSousa, S. M. (2001) Novel Scintillation Proximity Assay for Measuring Membrane-Associated Steps of Peptidoglycan Biosynthesis in Escherichia coli. Antimicrob. Agents Chemother. 45, 768-775.).

### SUMMARY OF THE INVENTION

The present invention employs hydrophobic resin beads as a means to capture hydrophobic reaction products and thereby provides a precise, high throughput method for the assay of many types of polyprenyl phosphate: hexosamine 1-phosphate transferases. These enzymes are often referred to herein as glycosyl transferases. The bead format assay can be used to detect or quantitate the levels of glycosyl transferase enzyme typically present in a broad range of natural and recombinant bacterial membrane extracts. The assay is exemplified for MraY, MurG and WecA and can be generalized to other related glycosyl transferases by utilizing diverse sugar-nucleotides as donors. The present invention thus provides the working basis for drug screening as well as detailed mutagenesis and enzymologic studies of this widespread and important protein family.

An aspect of this invention is an assay useful for detecting the attachment of sugar to polyprenyl phosphate carrier to form a polyprenyl phosphate-linked product. In general, it is preferred that a label, conveniently a radiolabel, is incorporated into a nucleotide sugar precursor. The labeled nucleotide sugar is added to a sample containing a polyprenyl phosphate carrier and bacterial glycosyl transferase. The transferase links the labeled sugar to the carrier to produce a labeled polyprenyl phosphate-linked product. Hydrophobic beads are added to the assay to capture the product. After separating the beads from unincorporated labeled nucleotide sugar, one can detect the labeled polyprenyl phosphate-linked product captured on the beads.

This aspect of the invention is adaptable to improve existing assays for the detection of the incorporation of a labeled nucleotide sugar into a labeled polyprenol phosphate-linked product. To adapt this invention to existing assays, one captures the labeled polyprenol phosphate-linked product on hydrophobic beads, separates the beads from unincorporated labeled nucleotide sugar and detects the presence of the labeled polyprenol phosphate-linked product on the beads.

In a particular embodiment of the invention, the labeled nucleotide sugar is UDP-MurNAc-[C¹⁴]pentapeptide, the polyprenyl phosphate carrier is decaprenol phosphate, the sample contains a bacterial MraY and the hydrophobic beads are HP20ss beads.

In a particular embodiment of the invention, the labeled nucleotide sugar is UDP-GlcN-[C¹⁴]Ac, the polyprenyl phosphate carrier is decaprenol phosphate, the sample contains bacterial MraY and bacterial MurG and the hydrophobic beads are HP20ss beads. In this embodiment, the assay in conducted in the presence of unlabeled UDP-MurNAc-pentapeptide and Triton X-100.

In a particular embodiment of the invention, the labeled nucleotide sugar is UDP-GlcN-[C¹⁴]Ac, the polyprenyl phosphate carrier is decaprenol phosphate, the sample contains bacterial WecA and the hydrophobic beads are HP20ss beads. In this embodiment, the assay is conducted in the presence of unlabeled UDP-MurNAc-pentapeptide and CHAPS.

The abbreviations used herein are: diaminopimelic acid, A₂pm; HPLC, high performance liquid chromatography; IPTG, isopropyl-β-D-thiogalactopyranoside; Lipid II, Bactoprenyl-pyrophosphoryl-MurNAc-pentapeptide; Lipid II, Bactoprenyl-pyrophosphoryl-MurNAc-pentapeptide-GlcNAc; PCR, polymerase chain reaction; UDP-MurNAc-tripeptide (meso-diaminopimelate-containing), UDP-MurNAc-L-Ala-γ-D-Glu-meso A₂pm; UDP-MurNAc-pentapeptide, UDP-MurNAc-L-Ala-γ-D-Glu-meso-A₂pm-D-Ala-D-Ala.

### BRIEF DESCRIPTION OF THE DRAWINGS

FIG. 1. Bacterial polyprenyl phosphate transferase reactions catalyzed at the cytoplasmic membrane. Gram negative cells initiate the production of enterobacterial common antigen by WecA-mediated transfer of GlcNAc 1-P from UDP-GlcNAc to bactoprenol monophosphate. In both Gram-positive and Gram-negative cells, the reactions catalyzed by the bacterial proteins MraY and MurG produce the lipid-linked precursors Lipid I and Lipid II respectively. In vitro assays utilize decaprenol phosphate as a surrogate for bactoprenol phosphate, represented by a wavy line.

FIG. 2. Schematic of exemplary reactions assayable by this bead-based system. All represented reactions catalyze the transfer of a water soluble radiolabeled-substrate to the lipophilic acceptor decaprenol phosphate. Panels A and C. Steps of stage II cell wall synthesis. MraY catalyzed 1-phospho-MurNAc-pentapeptide transfer and GlcNAc 1-P transfer catalyzed by MurG. The latter is measured as a coupled reaction; Panel B. Simple hexosamine 1-P transferase reaction exemplified by the E. coli WecA protein; Panel D. Format of the bead assay. No transfer steps occur. Reactions are performed directly in the wells of a 96-well filter plate. The flow of events for a single well is depicted. Reaction is halted with a pH drop, followed by adsorption of product onto solid phase beads. The unincorporated radiolabel is then washed from the beads which are then quantitated by scintillation spectrometry in situ.

FIG. 3. Verification of the lipid product quantitated in the MraY assay by TLC. Cellulose TLC plates (Merck Darmstadt) were developed in isobutyric acid: 1 M ammonium hydroxide (5:3 v/v), dried and imaged by phosphoimager. The origin and solvent front are marked. Panel A: HP20 beads were extracted with acetonitrile after processing in a MraY assay. The extract was spotted (lane 3) next to the butanol extract of both a standard MraY reaction (lane 2) and a no enzyme control (lane 1). One spot was seen at high Rf coincident with the standard reaction verifying that the beads quantitate the same product observed by butanol extraction. Panel B: Standards spotted for comparison. Starting material for the MraY assay, UDP-MurNAc-[¹⁴C]pentapaptide (lane 1) and Lipid I from the MraY reaction (lane 2), isolated as described in EXAMPLES V-VIII.

FIG. 4. Assay linearities. Assays were performed using extracts of E. coli strain MB2884. The operation of the assay is described in EXAMPLES V-VIII. Right panels show linearity with protein at 20 minutes time for Murein enzymes MraY and MurG and 10 minutes time for WecA. Panel A (left): Linearity of MraY assay with time at (^{•}) 0.25 mg/mL and (o) 0.125 mg/mL of membrane extract. Panel B (left): Linearity of MurG synthesis, detected by coupled assay as shown in FIG. 1 at (open triangles) 1.0 mg/mL and (^{•}) 0.5 mg/mL of membrane extract. Panel C (left): GlcNAc transfer to decaprenol phosphate catalyzed by WecA. Linearity of enzyme with time at (filled triangle) 0.5 mg/mL (o) 0.25 mg/mL and (^{•}) 0.125 mg/mL of membrane extract.

FIG. 5. Titration of MraY with the inhibitor tunicamycin and the MraY/MurG couple with ramoplainin. Panel A: Titration of the MraY assay with the inhibitor tunicamycin using 0.5 mg/mL protein at 10 min time. IC₅₀ is 1.1 μM +/− 0.08 μM. Panel B: Titration of the MraY/MurG couple with the MurG inhibitor ramoplainin using 0.5 mg/mL protein at 10 min time. IC₅₀ is 17 μg/mL+/1.1 μg/mL. These IC₅₀ₛ are consistent with literature reports (Brandish, P. E., Kimura, K.-I., Inukai, M., Southgate, R., Lonsdale, J. T., and Bugg, T. D. H. (1996) Modes of Action of Tunicamycin, Liposidomycin B, and Mureidomycin A: Inhibition of Phospho-N-Acetylmuramyl-Pentapeptide Translocase from Escherichia coli. Antimicrob. Agents Chemother. 40, 1640-1644; Somner, E. A. and Reynolds, P. E. (1990) Inhibition of Peptidoglycan Biosynthesis by Ramoplanin. Antimicrob. Agents Chemother. 34, 413-419).

### DETAILED DESCRIPTION OF THE INVENTION

The present invention provides a flexible assay system that is capable of measuring hydrophobic reaction products. The assay format is easily adaptable to a multi-well filter plate setting, can be quantitative and performed inexpensively. Particular assays can be designed to be specific and applicable to the wild-type levels of enzymes found in simple membrane extracts made from a range of common microorganisms. Using commonly and openly available materials, embodiments of this invention provide a generalizable, high throughput, one-pot assay for polyprenyl phosphate transferases by incorporating a solid-liquid, bead-based separation system to selectively adsorb the highly hydrophobic products of the reaction.

In a particular embodiments, described in detail herein, the bead format is applied to assay the formation of the MraY reaction product, the coupled MraY-MurG reaction product and the WecA reaction product. These embodiments demonstrate that this invention can be applied to measure not only activity of the translocase family members directly, but also enzymes that further modify the products of these transferases such as the glycosyl transferases of glycoprotein synthesis or O-antigen synthesis.

For example, the invention described herein can be generalized to any glycosyl transferase that utilizes a water soluble nucleotide-sugar precursor, obtainable in radio-labeled form, as substrate and creates a hydrophobic, polyprenol phosphate-linked product, either by direct linkage to this lipid carrier or by indirect linkage through existing carbohydrate moieties in this substrate. Examples are, but are not limited to, nucleotide-sugar transferases of Gram-negative LPS-linked core sugar assembly and O-linked outer antigen assembly, lipooligosaccharide assembly in Haemophilus and Meningitidus spp., Gram positive teichioic acid polymer synthesis, arabanomannan synthesis of tuberculosis spp., and glycan-based capsule formation.

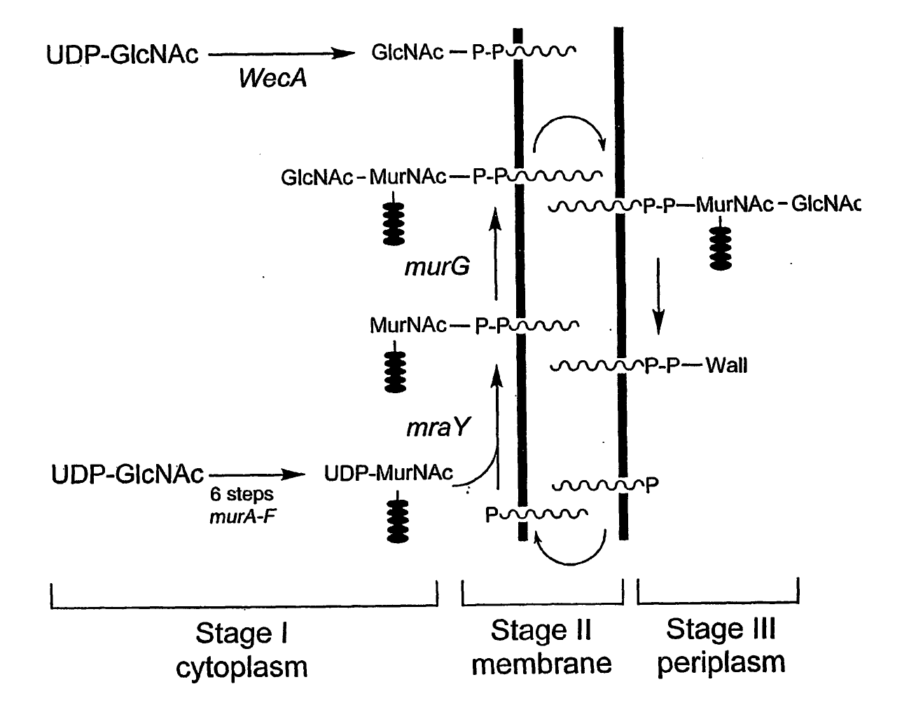

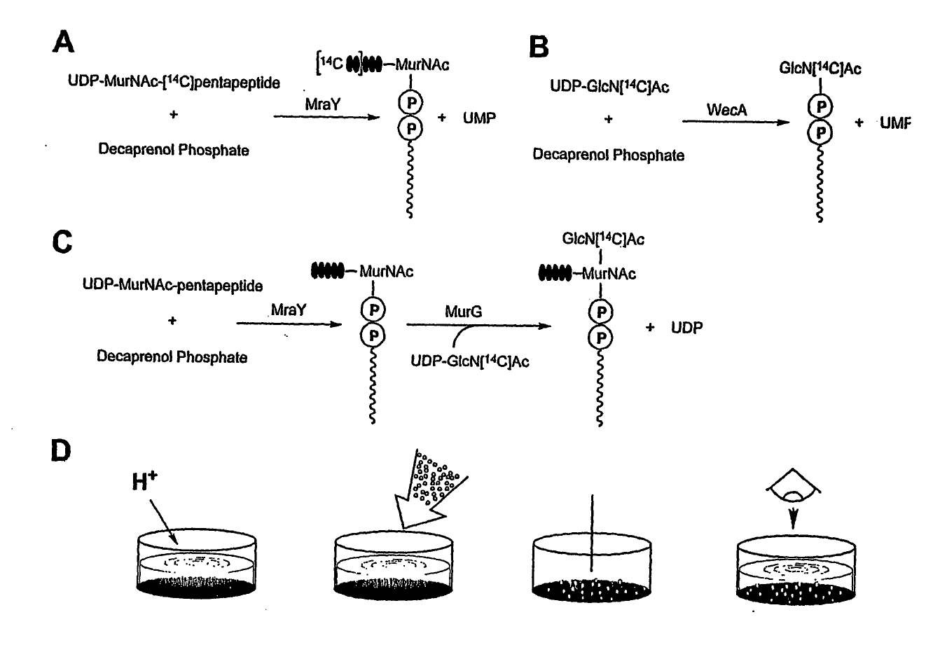

Conversion of liquid-liquid extraction assays to a solid-liquid format—The second stage of cell wall synthesis begins with the translocation of the soluble precursor, UDP-MurNAc-pentapeptide, to bactoprenol phosphate (FIG. 1). This lipid-linked intermediate is then further elaborated with an additional GlcNAc residue by the MurG protein. In each of these reactions, a soluble precursor is converted into a highly hydrophobic product, a property on which liquid-liquid extraction assays for these enzymes are based. The present invention reformulates this extraction step into a solid-liquid format using a suitably chosen hydrophobic bead. The resulting assay system can be easily manipulated in a filter plate setting allowing for the rapid examination of many samples simultaneously (FIG. 2, Panel D).

A range of commercially available hydrophobic resins can be used as long as the resin satisfies the user's criteria for, e.g., product capture, selectivity for the lipid linked product, retention of this product on the bead through wash steps, quantifiability in scintillation cocktail, ease of handling and cost. Of the resins tested for the particular assays exemplified herein, HP20 was found to bind the lipid-linked reaction products with high selectivity and with excellent retention through wash procedures. One should expect that some resins may bind product incompletely (polyamide in the exemplary assays) while others may not cleanly discriminate between substrate and product (SP207, C18 in the exemplary assays). Indeed, binding of the lipid-linked product to the HP20 beads was unaffected by the presence of the detergents used in the assay itself; no critical balance between detergent concentration and product recovery was observed. In broader application, one can follow the teachings and exemplifications herein to test a variety of resin beads in a variety of assays and determine which will perform appropriately to bind hydrophobic reaction products.

A mass amount of beads per well should be chosen that results in adequate product capture, good filtration rates and yield similar quantitation to a standard extraction assay run in parallel for comparison. One should also be aware that some beads may have a propensity settle rapidly, thereby resulting in inconsistent additions of bead per well and thus introducing a source of assay variation. However, the use of a microsized version of beads (HP20ss vs. HP20) or agitation may also address problems encountered due to the settling of beads.

MraY translocase assayed by bead format—Assay conditions and concentrations of metabolites were identical to those used by Brandish and co-workers in an extraction format without further optimization (Brandish, P. E., Burnham, M. K., Lonsdale, J. T., Southgate, R., Inukai, M., and Bugg, T. D. H. (1996) Slow Binding Inhibition of Phospho-N-acetylmuramyl-pentapeptide translocase (Escherichia coli) by Mureidomycin A. J. Biol. Chem. 271, 7609-7614.). By extraction of the product through the use of hydrophobic beads rather than an organic phase, one is able to increase sample throughput by minimizing material handling. However, the organic extraction step of Brandish et al., effectively stopped the reactions, which simple addition of the beads to the samples did not. Thus, one could incorporate an appropriate step to stop the reaction before adding capture beads. For example, for a Brandish et al., type assay, a step is incorporated to drop the pH to 2.0 in order to completely halt the reaction prior to bead addition. The products of this reaction were stable at this pH for several hours and no interference with binding of radiolabeled product to the beads was observed.

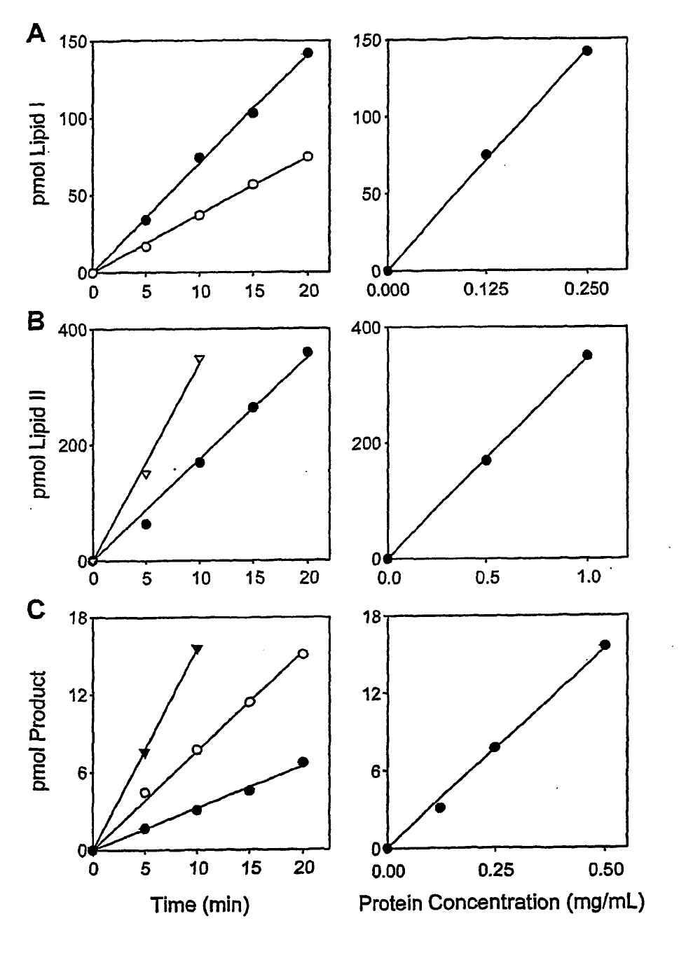

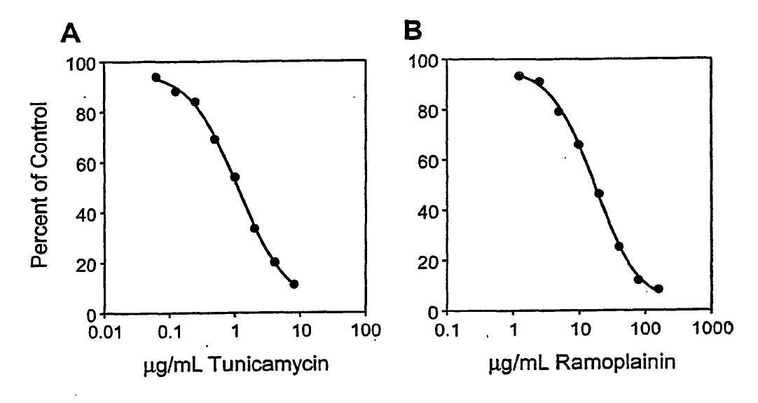

In the bead format assay of this invention, MraY activity measured in membranes prepared from wild-type E. coli was linear with both time and protein (FIG. 4, Panel A). This activity was inhibitable by the well described MraY inhibitor, tunicamycin, in a dose dependent manner (FIG. 5, Panel A). The IC₅₀ of approximately 1 μg/mL was consistent with literature reports for inhibition of cell wall precursor synthesis in broken cell systems (Brandish, P. E., Kimura, K.-I., Inukai, M., Southgate, R., Lonsdale, J. T., and Bugg, T. D. H. (1996) Modes of Action of Tunicamycin, Liposidomycin B, and Mureidomycin A: Inhibition of Phospho-N-Acetylmuramyl-Pentapeptide Translocase from Escherichia coli. Antimicrob. Agents Chemother. 40, 1640-1644.). DPM backgrounds in this system were typically 5% of control values allowing good quantitation of even highly inhibited samples.

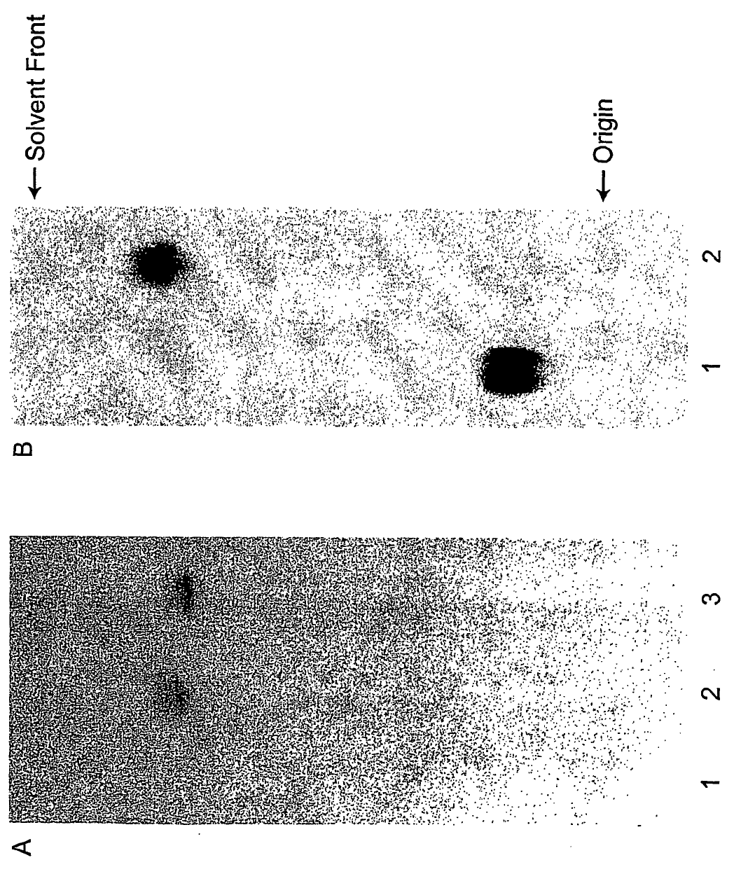

In order to confirm the nature of the quantitated product, the radioactivity captured by the HP20ss beads was extracted therefrom using acetonitrile. A majority of bead bound radioactivity was recovered and analyzed by thin layer chromatography alongside the pyridinium-acetate layer of a control (FIG. 3, Panel A, Lanes 2 and 3). In each case, a single band, distinct from the starting material (FIG. 3, Panel B Lane 1), was observed which migrated at the Rf of more rigorously isolated product (FIG. 3, Panel B, Lane 2).

Coupled assay of MraY and MurG—The ability of beads of HP20ss resin to adsorb the Lipid I generated in the MraY assay mixture was adequately demonstrated. To perform a combined assay, the use of these resin beads in quantitating Lipid II, the product generated by the MurG protein, would be useful. Accordingly, by coupling the endogenous MraY and MurG protein activities resident in an E. coli membrane extract, radiolabeled UDP-GlcN[1-¹⁴C]Ac was transferred to a more hydrophobic product, Lipid II, when incubated with extract in the presence of unlabeled UDP-MurNAc-pentapeptide and decaprenol phosphate. In this assay setting, it was convenient to increase the concentration of UDP-MurNAc-pentapeptide to 100 μM, both to create sufficient Lipid I to sustain the MurG-catalyzed reaction and to desensitize the assay to sequestration of this substrate. This activity was dependent on the addition of both UDP-MurNAc-pentapeptide and decaprenol phosphate.

Although more polar than Lipid I, the Lipid II formed in this reaction was also adsorbed by the HP20ss resin in a linearly time and protein dependent manner (FIG. 4, Panel B). Further, this activity was inhibitable by ramoplanin, a known MurG inhibitor, in a dose dependent fashion (FIG. 5, Panel B). The IC50 of approximately 17 μg/mL was consistent with literature reports for inhibition of cell wall precursor synthesis in broken cell or solubilized systems (Somner, E. A. and Reynolds, P. E. (1990) Inhibition of Peptidoglycan Biosynthesis by Ramoplanin. Antimicrob. Agents Chemother. 34, 413-419.). As in the direct assay for MraY, background DPM were less than 5% of control values.

The MraY protein is the only enzyme in bacteria that catalyzes transfer of MurNAc-1-phosphate-pentapeptide to a lipid-linked acceptor, thus ensuring the specificity of this assay for MraY activity. However, the substrate used to monitor the activity of MurG, UDP-GlcNAc, is also utilized by the WecA protein which catalyzes the addition of GlcNAc 1-phosphate directly onto bactoprenol phosphate as in enterobacterial common antigen synthesis (Rush, J. S., Rick, P. D. and Waechter, C. J. (1997) Polyisoprenyl Phosphate specificity of UDP-GlcNAc:undecaprenyl phosphate N-acetylglucosaminyl 1-P transferase from E. coli. Glycobiology 7, 315-322.) (FIG. 1). A priori, this would make it impossible for the bead extraction format to cleanly distinguish between these activities when using crude membrane extracts. Yet, specificity of this assay format for each of these enzymes can be achieved due to the distinct and non-overlapping detergent preferences of the WecA and MurG proteins: WecA is inactive in the Triton X-100 used to assay both the MraY and MurG proteins (Rush, J. S., Rick, P. D. and Waechter, C. J. (1997) Polyisoprenyl Phosphate specificity of UDP-GlcNAc:undecaprenyl phosphate N-acetylglucosaminyl 1-P transferase from E. coli. Glycobiology 7, 315-322.). The opposite is true of membranes assayed in the presence of CHAPS.

Both the direct MraY assay and the coupled MraY/MurG assay system functioned well to measure the corresponding activities in Staphylococcus aureus and Pseudomonas aeruginosa membranes, prepared in analogous fashion from wild-type to E. coli membranes. Interestingly, the observed specific activity of MraY as measured in the direct assay of similarly prepared membranes from each of these organisms, (E. coli: 111 pmol×min⁻¹×mg⁻¹, S. aureus: 104 pmol×min⁻¹×mg⁻¹, P. aeruginosa: 116 pmol×min⁻¹×mg⁻¹) was within 15 percent under these conditions.

Assay of WecA by bead format; extension of the bead principle—WecA protein activity was also assayable by this paradigm as described in EXAMPLE IX. The format required simply changing the substrates used and the detergent with which membranes were assayed. The WecA activity observed was dependent on the use of CHAPS as detergent with no significant activity detectable in the presence of Triton X-100.

WecA activity was linear with time and protein (FIG. 4, Panel C). Further verification that the source of the product measured in this assay was produced only by the WecA protein was seen when the activity of membranes derived from E. coli strain 21548(DE3)(WecA::Tn10, T7 polymerase lysogen) and from this strain bearing a plasmid engineered to overexpress WecA were assayed. The mutant, which has been demonstrated to be completely deficient in wecA-encoded transferase by paper chromatography assay (Meier-Dieter, U., Starman, R., Barr, K., Mayer, R. and Rick, P. D. (1990) Biosynthesis of Enterobacterial Common Antigen in Escherichia coli Biochemical characterization of Tn10 insertion mutants defective in enterbacterial common antigen synthesis. J. Biol. Chem 265, 13490-13497.), gave no detectable signal in the bead system (Table 1). However, the overexpressing variant gave a 70-fold enhanced signal compared to a wild-type strain tested in parallel, thus confirming the ability of the assay to quantitate WecA activity specifically. As in the MraY and MraY/MurG coupled assays, the background DPM were not more than 5% of control.

Many of the nucleotide-sugars used in these particular assays are currently available in radiolabeled form. Less common sugar-nucleotides will be necessary in order to assay more unusual transferases. Such radiolabeled materials could be obtained by classic techniques and should become increasingly available for common use as the synthetic routes to these nucleotide-sugars are revealed through emerging functional genomics. Such pathways could be exploited to achieve the semisynthetic synthesis of these precursors as has been reported for UDP-GlcN[1-¹⁴C]Ac (Leiting, B., Pryor, K-A. D., Eveland, S. S. and Anderson, M. S. (1998) One-day enzymatic synthesis and purification of UDP-N-[1-¹⁴C]acetyl-glucosamine. Anal. Biochem. 256, 185-191.). Such novel radiotracers, used in conjunction with the adaptable bead assay format described, allow an efficient and thorough examination of the enzymologic basis for substrate specificity and catalysis utilized by each of the polyprenyl phosphate transferase subspecies.

One of skill in the art will recognize that the description above and Examples below provide particular instances in which the solid-liquid, bead capture format of the present invention can be adapted to particular assays. These particular instances are exemplary and not meant as limiting of the scope of the invention.

### EXAMPLE I

Materials and Strains

Materials—UDP-[6-³H]-GlcNAc was obtained from AMERSHAM PHARMACIA BIOTECH INC. (Piscataway, N.J.). The preparation of UDP-N-[1-¹⁴C]acetyl-glucosamine is known in the art (Leiting, B., Pryor, K-A. D., Eveland, S. S. and Anderson, M. S. (1998) One-day enzymatic synthesis and purification of UDP-N-[1-¹⁴C]acetyl-glucosamine. Anal. Biochem. 256, 185-191.). Polyamide, XAD-2, SP207, SP800, HP20, HP20ss and C18 resins are available or were obtained from SUPELCO (Bellefonte, Pa.).

Prior to use, all resins were washed extensively with acetone, followed by methanol. The resins were then washed extensively with water and stored at room temperature. Decaprenol phosphate and phosphatidyl glycerol were from SIGMA ALDRICH (St. Louis, Mo.). Prenyl phosphates are also available from INDOFINE CHEMICAL COMPANY, INC. (Sommerville, N.J.). UDP-MurNAc-tripeptide (meso-A₂pm-containing) was isolated from Bacillus megaterium (Nakatani, T., Araki, Y. and Ito, E. (1968) Preparation and characterization of uridine diphosphate-N-acetylmuramyl-L-alanyl-D-glutamyl-meso-2,6-diaminopimelic acid. Biochim. Biophys. Acta. 156, 210-212.). UDP-MurNAc-tripeptide (L-lysine-containing) was isolated from Staphylococcus aureus in the same manner. Remaining minor impurities were removed using the reversed phase BPLC system of Flouret et al. (Flouret, B., Mengin-Lecreulx, D., and van Heijenoort, J. (1981) Reverse-phase high-pressure liquid chromatography of uridine diphosphate N-acetylmuramyl peptide precursors of bacterial cell wall peptidoglycan. Anal. Biochem. 114, 59-63.).

Bacterial strains—In general, strains of bacteria are appropriate for use in the assays of MraY, MurG and WecA provided the strain is not mutated at the genetic loci encoding the relevant protein resulting in no expression of the protein or expression of an inactive form of the protein. The following strains are all of Escherichia coli. Strain BL21 was purchased from NOVAGEN (Madison, Wis.). XL 1 Blue (recA⁻) was obtained from STRATAGENE (La Jolla, Calif.). E. coli strain MB2884 was obtained from the Merck Clinical Collection. E. coli strain 21548 (WecA::Tn10) (Meier-Dieter, U., Starman, R., Barr, K., Mayer, R. and Rick, P. D. (1990) Biosynthesis of Enterobacterial Common Antigen in Escherichia coli Biochemical characterization of Tn10 insertion mutants defective in enterbacterial common antigen synthesis. J. Biol. Chem 265, 13490-13497.) was the generous gift of Dr. Paul Rick, Uniformed Services of the Health Sciences, Bethesda, Md. For expression of the pET series of T7 promoter based-plasmids, this strain was converted into a DE3 lysogen using the lambda DE3 LYSOGENIZATION KIT (NOVAGEN, Madison, Wis.) as directed by the manufacturer. One candidate lysogen showing low basal expression of T7 polymerase, yet capable of high level induction with IPTG was chosen and named 21548(DE3).

Wild type Pseudomonas aeruginosa strain MB3286 and Staphylococcus aureus strain MB4447 were obtained from the Merck culture collection. However, other strains of these bacteria are suitable for assays provided the strain is not mutated at the relevant genetic loci encoding the relevant protein resulting in no expression of the protein or expression of an inactive form of the protein.

### EXAMPLE II

Preparation of Bacterial Membranes

E. coli bacterial membranes were prepared from cultures in LB (Luria-Bertani) broth (10 g tryptone, 5 g yeast extract, 10 g NaCl per liter). The cultures were incubated at 300 r.p.m. and 37° C. to late log phase. Cells were harvested by centrifugation at 8,000×g for 10 min and washed once in one volume of 10 mM potassium phosphate, pH 7.0. Cells were resuspended in about one twentieth the volume of the same buffer and broken in a French pressure cell at 18,000 p.s.i. Debris and unbroken cells were then removed by centrifugation at 20,000×g for 10 min. The membrane fraction was produced by centrifugation of the clarified supernatant at 100,000×g for 60 minutes. After removal of the soluble material, the membranes were resuspended in 50 mM Tris-Cl containing 0.1 mM MgCl₂ at pH 8.0. Membranes of P. aeruginosa and S. aureus were prepared, as described above, from cells grown in LB broth. Protein concentrations were determined by the method of Smith et al. (Smith, P. K., Krohn, R. I., Hermanson, G. T., Mallia, A. K., Gartner, F. H., Provenzano, M. D., Fujimoto, E. K., Goeke, N. M., Olson B. J., and Klenk, D. C. (1985) Measurement of protein using bicinchoninic acid Anal. Biochem. 150, 76-85.) using bovine serum albumin as a standard.

### EXAMPLE III

Cloning of wecA from Escherichia Coli

The E. coli wecA gene was retrieved by PCR from E. coli strain MB2884 using the genomic DNA sequence from Accession Number M76129. The final clone, pWecA, initiated at the same. location as pAA14 of Amer and Valvano (Amer, A. O., and Valvano, M. A. (2000) The N-terminal region of the Escherichia coli WecA (Rfe) protein, containing three predicted transmembrane helices, is required for function but not for membrane insertion. J. Bacteriol. 182, 498-503.), who reported that the most upstream putative start site, homologous to other WecA bacterial sequences, is the native start site of the wecA reading frame. PCR primers were designed to allow unidirectional ligation of resulting PCR fragments into the vector pET11a. Primers were (forward): 5′-GCGCGC ATCGTA CATATG AATTTA CIGACA GTGAGT ACTG-3′ (SEQ ID NO:1) and (reverse): 5′-CGCGCG ATCGTA GATCTT CATTAT TTGGTT AAATTG GGGCTG CC-3′(SEQ ID NO:2). Each primer incorporated a G/C clamp and a restriction site; a Nde I site immediately 5′ of the initiating ATG codon in the forward primer and a BglII site immediately 3′ of tandem stop codons in the reverse primer. PCR reactions contained 200 ng of genomic DNA, 1 μM of each primer, 200 μM dNTP's, and 2.5 units of PFU DNA polymerase in 20 mM Tris-HCl (pH 8.75), 10 mM KCl, 10 mM (NH₄)₂SO₄, 2 mM MgSO₄, 0.1% Triton X-100, and 100 μg/mL bovine serum albumin. Thermocycling was performed in 35 cycles according to the schedule: 30 s at 97° C. melting, 30 s annealing at 61° C. and 2 min at 72° C. elongation. All reactions were polished at 72° C. for 10 min prior to further use. The resulting PCR product was purified using the QIAPREP SPIN MINIPREP KIT (QIAGEN INC., Valencia, Calif.), digested sequentially with Nde I and Bam HI, and ligated into similarly digested pET11a vector. Transformation of the ligation reaction into electroporation-competent EPICURIAN COLI XL-1 BLUE cells (STRATAGENE, La Jolla, Calif.) yielded ampicillin resistant colonies. Putative pWecA plasmids were identified by restriction digestion and verified by direct DNA sequencing. Isolates from two separate PCR reactions were cloned to avoid any variations in sequence derived from PCR errors (Accession Number AF248031).

### EXAMPLE IV

Expression of pET-Based Plasmids in E. Coli and Preparation of Bacterial Membranes.

E. coli strain 21548(DE3) was made electrocompetent and transformed simultaneously with the chloramphenicol resistant plasmid pLysS (25 μg/1 mL) and each of the ampicillin resistant pET11-based plasmids pET11a and pWecA, (100 μg/mL). Cultures of cells (125 mL) harboring the expression plasmids were incubated in 500 mL flasks at 300 rpm and 37° C. in LB medium containing ampicillin (100 μg/mL) and chloramphenicol (25 μg/mL) until the culture reached an OD₆₀₀ of 0.7. Expression of each protein was induced with 1 mM IPTG for 2 h. The cells were harvested by centrifugation (8,000×g for 10 min at 4° C.), resuspended in 2.5 mL of 10 mM potassium phosphate, pH 7.0, and lysed at 18,000 p.s.i. using a French pressure cell. Cellular debris was removed by centrifugation 20,000×g for 10 min at 4° C.). The supernatant was collected and recentrifuged (100,000×g for 1 h at 4° C.) in order to collect membranes. After removal of the soluble material, the membranes were resuspended in 50 mM Tris-Cl containing 0.1 mM MgCl₂, pH 8.0, with the aid of a Potter Helvejehm homogenizer mortar, analyzed for protein content by the bicinchoninic acid method (Smith, P. K., Krohn, R. I., Hermanson, G. T., Mallia, A. K., Gartner, F. H., Provenzano, M. D., Fujimoto, E. K., Goeke, N. M., Olson B. J., and Klenk, D. C. (1985) Measurement of protein using bicinchoninic acid Anal. Biochem. 150, 76-85.) and stored flash frozen in aliquots at −80° C.

### EXAMPLE V

Extraction Assay for MraY (UDP-MurNAc-pentapeptide Translocase)

The translocase was alternatively assayed using the pyridinium-acetate extraction procedure of Brandish et. al. (Brandish, P. E., Burnham, M. K., Lonsdale, J. T., Southgate, R., Inukai, M., and Bugg, T. D. H. (1996) Slow Binding Inhibition of Phospho-N-acetylmuramyl-pentapeptide translocase (Escherichia coli) by Mureidomycin A. J. Biol. Chem. 271, 7609-7614.).

Preparation of UDP-MurNAc-[¹⁴C]pentapeptide—This murein precursor was synthesized from [1-¹⁴C]D-Alanine and UDP-MurNAc-tripeptide using the enzymes Dd1B and MurP, each supplied as a fusion protein with glutathione S-transferase (Anderson, M. S., Eveland, S. S., Onishi, H. R., and Pompliano, D. L. (1996) Kinetic mechanism of the Escherichia coli UDP-MurNAc-tripeptide D-alanyl-D-alanine-adding enzyme: use of a glutathione S-transferase fusion. Biochemistry 35, 16264-16269.). Briefly, synthetic reactions contained Tris, pH 8.6 (100 mM), KCl (40 mM), MgCl₂ (10 mM), NaCl (500 mM), ATP (16 mM), UDP-MurNAc-tripeptide (2.23 mM), [1-14C]D-Alanine (43 mCi/mmol; 0.0208 microcuries/mL; 0.466 mM), GST:MurF (0.113 mg/mL) and GST::Dd1B (0.012 mg/mL) in a volume of 2.5 mL. Reaction occurred at 25° C. for 18 h. The reaction was simplified for HPLC purification by dilution 10-fold with water and passage over 0.8 mL DOWEX AG1-X2, chloride form. The column was washed with 5 column volumes of 50 mM ammonium formate, pH 4.0, and both the UDP-MurNAc-tripeptide and UDP-MurNAc-[¹⁴C]pentapeptide were eluted with 5 column volumes of 1 M ammonium formate, pH 4.0. The bulk of ATP and ADP remained on the column. The eluate was diluted and lyophylized overnight to a powder. This sample was resuspended in 1 mL of 50 mM ammonium formate, pH 4.40, and purified on a 7.8 mm×250 mm ODS-HPLC column in four portions according to Flouret (Flouret, B., Mengin-Lecreulx, D., and van Heijenoort, J. (1981) Reverse-phase high-pressure liquid chromatography of uridine diphosphate N-acetylmuramyl peptide precursors of bacterial cell wall peptidoglycan. Anal. Biochem. 114, 59-63.). The product was collected and lyophylized. The final product was resuspended in 0.5 mL of water and quantitated by liquid scintillation spectrometry. Typically yields averaged 78% incorporation of the initial radiolabeled alanine into the UDP-MurNAc-pentapeptide. This preparation worked identically using either UDP-MurNAc-lysyl-tripeptide or UDP-MurNAc-diaminopimelate-tripeptide, consistent with the lack of selectivity imposed by the MurF protein with regard to these two substrates (Anderson, M. S., Eveland, S. S., Onishi, H. R., and Pompliano, D. L. (1996) Kinetic mechanism of the Escherichia coli UDP-MurNAc-tripeptide D-alanyl-D-alanine-adding enzyme: use of a glutathione S-transferase fusion. Biochemistry 35, 16264-16269.).

### EXAMPLE VI

Assessment of Optimal Assay Bead

Six polymeric bead types of varying hydrophobicity, Polyamide, XAD-2, HP20, C18, SP207 and SP800, were tested for their ability to bind and retain radiolabeled-Lipid I. This ligand was generated by allowing 1 mL of MraY assay cocktail (see below) to come to equilibrium for 30 min at 30° C. The resulting mixture of both product and UDP-MurNAc-[¹⁴C]pentapeptide was apportioned into 100 μL aliquots, each mixed with an equal volume of a different solvent prewashed resin that had been slurried 1:1 (v/v) in water. These tubes were mixed on a rotary wheel for 10 minutes at room temperature and the beads collected by 5 min centrifugation in a microcentrifuge. The supernatant was separated from the beads completely using a fine gauge pipette tip and quantitated by liquid scintillation spectrometry. The resin was then washed five times with 10 mM Bis-tris, pH 6.0. Each wash was collected and quantitated as above. Finally the resins were washed with acetonitrile in two portions which were also quantitated. Samples containing significant counts were analyzed further by thin layer chromatography on Cellulose TLC plates (Merck Darmstadt), developed with 5:3 (v/v) isobutyric acid/1 M ammonium hydroxide and imaged using a MOLECULAR DEVICES (Sunnyvale, Calif.) phosphoimaging station.

One aliquot of the reaction mixture was extracted with pyridinium acetate, pH 4.2, as performed in the standard extraction assay (Brandish, P. E., Burnham, M. K., Lonsdale, J. T., Southgate, R., Inukai, M., and Bugg, T. D. H. (1996) Slow Binding Inhibition of Phospho-N-acetylmuramyl-pentapeptide translocase (Escherichia coli) by Mureidomycin A. J. Biol. Chem. 271, 7609-7614.). The yield from this reaction was used as a 100% control for the bead analysis above and the organic phase was used as a TLC standard for the product.

### EXAMPLE VII

Assay of MraY Using HP20ss Hydrophobic Beads

The reaction catalyzed transfer and use of the radiotracer are shown in FIG. 2, Panel A. The general course of manipulations are diagramed in FIG. 2, Panel D and described in the text. The reaction mixture for the MraY assay included 100 mM Tris, pH 7.5, 30 mM MgCl₂, 0.15% Triton X-100, 100 μg/ml Phosphatidyl Glycerol, 40 μM decaprenol phosphate, 14 μM UDP-MurNAC-[¹⁴C]pentapeptide (prepared as described above) and typically 0.5 mg/mL of E. coli cell membranes in a final volume of 50 μL. The phosphatidyl glycerol and decaprenol phosphate, which are supplied in organic solvent, were dried in the reaction tube before the addition of the other components.

The HP20ss resin used to isolate the Lipid I product from the above reaction mixture was equilibrated before use. Briefly, the dry resin was mixed thoroughly with excess methanol in a beaker and allowed to settle for 45 min. The methanol was decanted along with fines and the procedure repeated six times with distilled water in order to remove all traces of methanol. After the final water wash, an equal volume of distilled water was added to the resin bed which could be stored at room temperature indefinitely.

For assays whereby the reaction is analyzed at various times, a larger reaction volume was prepared (50 μL is typically appropriate for each time point). The assay may be performed in microcentrifuge tubes, but for larger numbers of samples, we performed the reaction in the wells of a MHVB N45 filter plate (MILLIPORE, Bedford, Mass.) at room temperature. The membrane preparation was added last in order to initiate the reaction. For each time point, an aliquot (50 μL) was removed from the reaction mixture and transferred to a separate well in the filter plate containing 50 μL of 40 mM HCl. The acid treatment effectively terminates the reaction by lowering the pH to 2.0 One hundred microliters of pre-equilibrated and freshly flocculated HP20ss resin was then added to the terminated reactions using a repeater pipet. The filter plate was left to sit at room temperature for 20 min in order to allow the product of the reaction (¹⁴C-labeled lipid I) to bind to the beads. The liquid was then drawn from the beads using a vacuum manifold for filter plates. In order to remove residual unbound UDP-MurNAc-[¹⁴C]pentapeptide, the resin was washed with 200 μL of distilled water, delivered to the wells using a multichannel pipettor, followed by filtration on the vacuum manifold. This wash step was repeated through four cycles. Some care should be taken not to over-dry the beads which could prevent them from wetting properly again during the washing procedure. However, after the last wash, air was drawn through the filter plate for 20 sec in order to remove any remaining liquid from the resin. The filterplate was then quantitated by liquid scintillation spectrometry with a TOPCOUNT (PERKIN ELMER LIFE SCIENCES, Downers Grove, Ill.) using 200 μL of scintillation cocktail (MICROSCINT 40, PERKIN ELMER LIFE SCIENCES, Downers Grove, Ill.) per well.

### EXAMPLE VIII

Coupled Assay of MraY and MurG using HP20ss Beads

The reactions catalyzed and use of the radiotracer are shown in FIG. 2, Panel C. The MraY/MurG coupled assay reaction procedure was identical to that described above for the assay of MraY. However, quantitation of the coupled system was performed by monitoring the incorporation of radiolabeled MurG co-substrate UDP-N-[1-¹⁴C]acetyl-glucosamine into the lipophilic product Lipid II. Accordingly, this assay mixture was modified to contain 100 mM Tris, pH 7.5, 30 mM MgCl₂, 0.15% Triton X-100, 40 μM decaprenol phosphate, 100 μM UDP-MurNAc-pentapeptide, 100 ug/ml phosphatidyl glycerol, and 20 μM, 0.1 μCi UDP-N-[1-¹⁴C]acetyl-glucosamine and typically 0.5 mg/mL of E. coli cell membranes in a final volume of 50 μL. All other procedures, times and temperatures were identical to those described for the MraY assay.

Preparation of Lipid I—Lipid I could be prepared from a 250 μL MraY assay cocktail which had been allowed to react 30 min at room temperature in the presence of an MraY overproducing extract. To the reaction was added 250 μL of 6 M pyridinium-acetate, pH 4.2, and after vortexing, 500 μL of n-butanol followed by an additional 250 μL 6 M pyridinium-acetate, pH 4.2. The sample was vortexed and the layers separated by centrifugation in a microcentrifuge for 5 min. The upper butanol phase was removed carefully and placed in a separate tube. This phase was vortexed with 0.5 vol of deionized water and centrifuged for 5 min. The washed butanol phase was recovered, shell frozen in a 15 mL conical falcon tube and lyophylized for 20 min. Lyophylization progress should be monitored and the sample refrozen if necessary. As soon as the sample was dried, it was removed and resuspended in 100 μL of 40 mM bis-tris, pH 6.5, containing 0.15% Triton X-100 and immediately placed on ice. Material prepared in this manner showed no significant decomposition (FIG. 3, Panel B, lane 2) and was suitable as a substrate for the MurG reaction. This material could be stored at 4° C. for several days or frozen for weeks.

### EXAMPLE IX

Assay of WecA Using HP20ss Beads

The reaction catalyzed transfer and the use of the radiotracer are shown in FIG. 2, Panel B). The reaction mixture for the WecA assay was essentially that of Rush et al., (Rush, J. S., Rick, P. D. and Waechter, C. J. (1997) Polyisoprenyl Phosphate specificity of UDP-GlcNAc:undecaprenyl phosphate N-acetylglucosaminyl 1-P transferase from E. coli. Glycobiology 7, 315-322.) used without further optimization and included 50 mM Tris, pH 8.0, 40 mM MgCl₂, 0.5 mM EDTA, 0.5% CHAPS, 50 mM sucrose, 5 mM beta-mercaptoethanol, 50 μM decaprenol phosphate and 28 μM, 0.14 μCi UDP-N-[1-¹⁴C]acetyl-glucosamine and typically 0.5 mg/ml of E. coli cell membranes in a final volume of 50 μL. The decaprenol phosphate was dried in the reaction tube and resuspended in the CHAPS detergent prior to the addition of other materials. The reaction was initiated by the addition of enzyme and was performed at 37° C. Acid quench, HP20ss bead extraction and quantitation procedures were as described for the MraY assay.

TABLE 1
WecA protein catalyzed GlcNAc-1-P transferase activity
as measured by bead assay in E. coli membrane
extracts from expressed plasmid constructs.

| | Spec. Activity | Relative |
|---|---|---|
| Host strain | pmol · min⁻¹ · mg⁻¹ | Activity |
| MB2884 (Wild Type) | 58 | 1 |
| (WecA null) | | |
| 21548(DE3)/ | | |
| pLysS | ND | <0.05 |
| pLysS/pET11a | ND | <0.05 |
| pLysS/pWecA | 4071 | 70 |

ND = Not detectable

What is claimed:

1. An assay for detecting the attachment of sugar to polyprenyl phosphate carrier to form a polyprenol phosphate-linked product comprising:
a) adding a labeled nucleotide sugar to a sample containing at least one bacterial glycosyl transferase and polyprenyl phosphate carrier,
b) allowing the incorporation of the labeled nucleotide sugar into a labeled polyprenol phosphate-linked product;
c) adding hydrophobic beads;
d) capturing the labeled polyprenol phosphate-linked product on the beads,
e) separating the beads from unincorporated labeled nucleotide sugar and
f) detecting the presence of the labeled polyprenol phosphate-linked product on the beads.

2. The assay according to claim 1 wherein the labeled nucleotide sugar is UDP-MurNAc-[C¹⁴]pentapeptide, the polyprenyl phosphate carrier is decaprenol phosphate, the sample contains a bacterial MraY and the hydrophobic beads are HP20ss beads.

3. The assay according to claim 1 wherein the labeled nucleotide sugar is UDP-GlcN-[C¹⁴]Ac, the polyprenyl phosphate carrier is decaprenol phosphate, the sample contains bacterial MraY and bacterial MurG and the hydrophobic beads are HP20ss beads, further comprising conducting the assay in the presence of unlabeled UDP-MurNAc-pentapeptide and Triton X-100.

4. The assay according to claim 1 wherein the labeled nucleotide sugar is UDP-GlcN-[C¹⁴]Ac, the polyprenyl phosphate carrier is decaprenol phosphate, the sample contains bacterial WecA and the hydrophobic beads are HP20ss beads, further comprising conducting the assay in the presence of unlabeled UDP-MurNAc-pentapeptide and CHAPS.

5. In an assay for the detection of the incorporation of a labeled nucleotide sugar into a labeled polyprenol phosphate-linked product, the improvement comprising capturing the labeled polyprenol phosphate-linked product on hydrophobic beads, separating the beads from unincorporated labeled nucleotide sugar and detecting the presence of the labeled polyprenol phosphate-linked product on the beads.


SEQUENCE LISTING (2 sequences)

SEQ ID NO: 1 - DNA, 40 bp, Eschericia coli
gcgcgcatcgtacatatgaatttactgacagtgagtactg

SEQ ID NO: 2 - DNA, 44 bp, Eschericia coli
cgcgcgatcgtagatcttcattatttggttaaattggggctgcc